(19) United States
(12) Patent Application Publication
(10) Pub. No.: US 2021/0059932 A1
(43) Pub. Date: Mar. 4, 2021

(54) Multi-layer edible strip containing active medicinal ingredients

(71) Applicant: Vincent Langlois, Blainville (CA)

(72) Inventor: Vincent Langlois, Blainville (CA)

(21) Appl. No.: 16/553,261

(22) Filed: Aug. 28, 2019

(51) Int. Cl. A61K 9/00; A61K 31/352; A61K 36/185; A61K 9/70; A23L 33/19; A23L 33/15

(52) U.S. Cl. CPC A61K 9/0056; A61K 31/352; A23L 33/15; A61K 9/7007; A23L 33/19; A61K 36/185

(57) ABSTRACT

In one embodiment of the present invention an improved recipe containing whey proteins and ascorbic acid is used to make three separate layers that are laid one of top of the other wherein the central layer is a separator layer used for separating the two outlying layers containing active medicinal components. The three layers, together, are thicker than what is currently possible and as such, contain more active ingredients, which is crucial for medicinal purpose.

### CROSS-REFERENCE TO RELATED APPLICATIONS

N/A

### BACKGROUND OF THE INVENTION

### 1. Field of the Invention

The present invention generally relates to medication delivery systems but more particularly to a multi-layer edible strip containing active medicinal ingredients.

### 2. Description of Related Art

The delivery of medicine to a patient is very important and depending upon the type of medicine, taking in medicine can vary from type to type. For example, some medicine come in liquid form while others come in solid form. Some medicines can be altered so as to be available in both liquid and solid forms. Other medicines need to be injected intravenously. The latest form of medicine consumption comes in the form of edible strips put on the tongue where they quickly dissolve in order to liberate the active ingredients. The most common use of this type of ingestion is in the Listerine™ breath strips. When attempts at using this technique for medication, it was found that those edible strips could not contain enough active ingredients for various physical reasons. Firstly, if too much active ingredients is admixed, the physical integrity of the strip, which is made mostly of Pullulan™, breaks down. Secondly, attempts at making the strip thicker so as to contain more medicine also failed. There is thus a need for improvement in this field.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In one aspect of the invention, a multi-layer edible strip is provided, comprising a first layer of active medicinal ingredients; a second layer of active medicinal ingredients; and, a third layer of a food film base, wherein the first layer and the second layer are separated by the third layer.

In one embodiment, the active medicinal ingredients include THC. In one embodiment, the multi-layer edible strip is approximately 45 microns to 150 microns. In one embodiment, the third layer of the food film base acts as an adhesive between the first and second layers of active medicinal ingredients. In another embodiment, soy lecithin is used as an emulsifying agent with the THC. In one embodiment, the multi-layer edible strip contains a modified starch. In another embodiment, the multi-layer edible strip contains whey protein isolate and ascorbic acid. In one embodiment, 15 to 25 mg of THC is comprised in the multi-layer edible strip.

In another aspect of the invention, a multi-layer edible strip is provided, consisting of a first layer of an ingredients formulation including THC; a second layer of an ingredients formulation including THC; and, a third layer of a food film base, wherein the first layer and the second layer are separated by and joined to the third layer.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a representation of a user pulling out a strip from a dispenser of the prior art.

FIGS. 2A-B are exemplary instances of active medical ingredients for multi-layer edible strips according to an embodiment of the present invention.

FIG. 3 is flow chart illustrating the fabrication process of the prior art.

FIG. 4 is a flow chart illustrating the fabrication process according to an embodiment of the present invention.

FIG. 5 is a multi-layer edible strip according to an embodiment of the present invention.

FIG. 6 is a schematic view of the production system of the prior art.

FIG. 7 is a schematic view of the production system according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide a multi-layer edible strip containing active medicinal ingredients.

The word “a” is defined to mean “at least one.” “THC” is defined to mean “tetrahydrocannabinol” as well as similar or related cannabinoids. The terminology includes the words above specifically mentioned, derivatives thereof, and words of similar import.

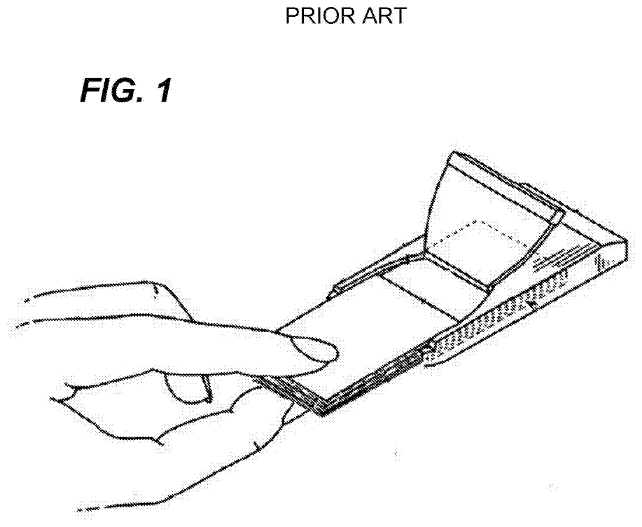

FIG. 1 is a representation of a user pulling out a strip from a dispenser of the prior art. Referring now to FIG. 1, the strips of the prior art are thinner than the strips of the present invention, thus, fewer strips can be inserted in the dispenser or, alternatively, a thicker dispenser is required to hold the same number of strips. All other manner of use is similar.

In the prior art, the strips contain soluble food films with active ingredients (THC) and are composed of 3 major groups: (a) plasticizers (“laminators”): made out of gums (carrageenan, xanthan, carob, arabica); (b) polymers (polysaccharides/pullulans), wherein the polymers (aminated amine branches of different glucose/glycerol) and the gums serve as plasticizers and ensures the stability, dissolvability and durability of the film structure; and (c) sweetening agents and flavors, including sweeteners like acesulfame-potassium, sucralose, stevia and aspartame, natural and/or artificial flavors and dyes, wherein the sweeteners are used for their high sugar concentration in a small density and dissolvability. The active agents (THC) include botanical extracts, vitamins, etc.

It is a particular objective of the present invention to solve the problem of having 20 mg of active agents per food film as well as overcoming six criteria of technological uncertainty with a similar assay that has never been introduced or developed to date, have been established such as: (a) consistency of the product; (b) durability (shelf Life, a desired parameter in the industry); (c) solvency of the finished product; (d) effectiveness (sufficient concentration of active agents in each strip); (e) texture (to allow proper packaging and meet solvency/uniformity criteria); and (f) taste.

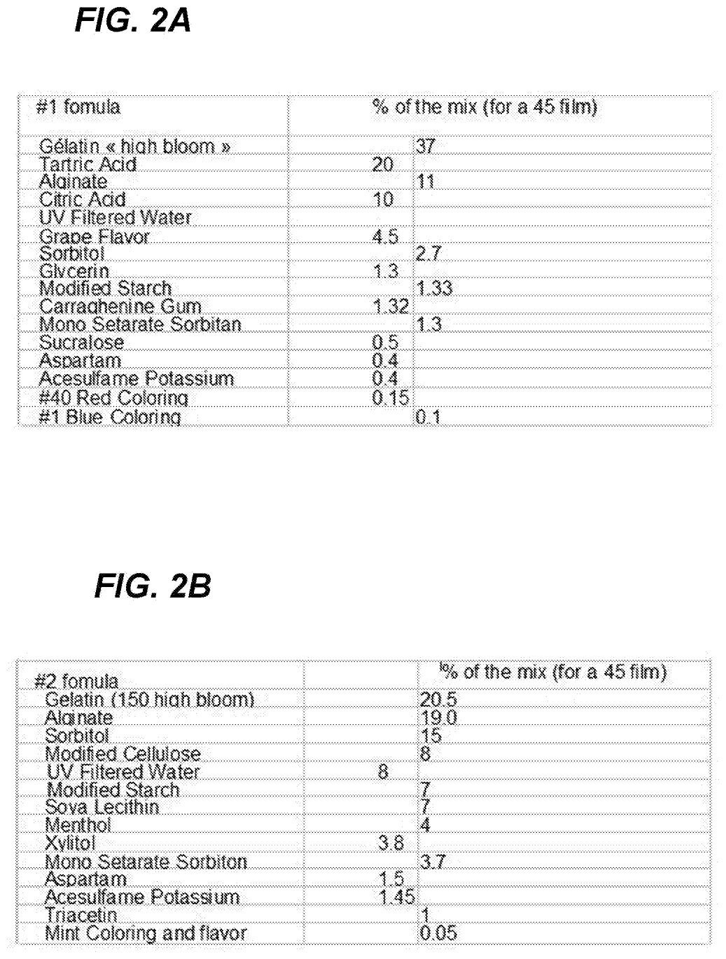

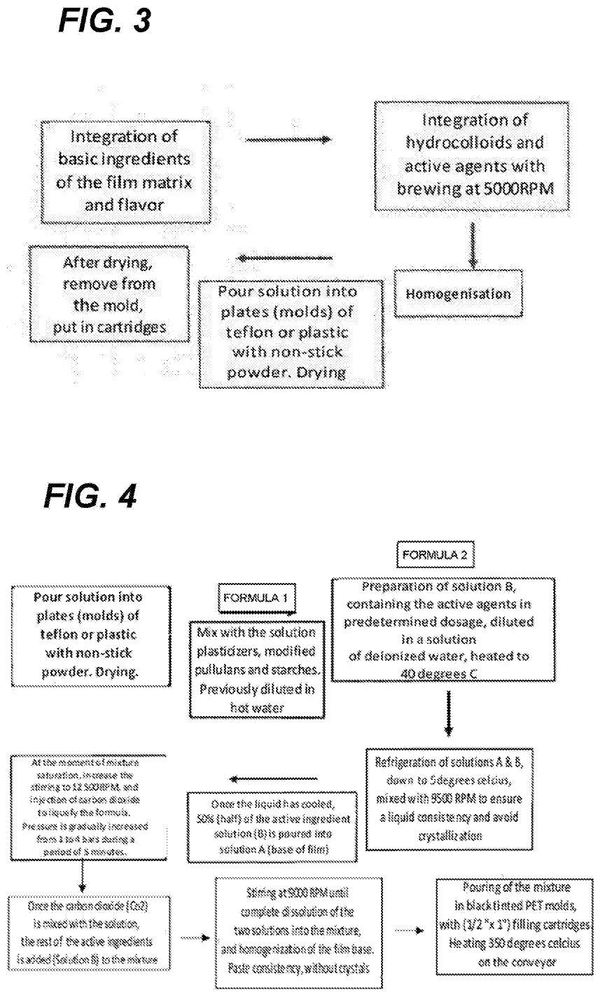

FIGS. 2A-B are exemplary instances of active medical ingredients for multi-layer edible strips according to an embodiment of the present invention. Referring now to FIGS. 2A-B, various recipe formulations or matrices are provided, which have been proven to be efficient. There are new elements in the film matrix which provide a more soluble film matrix while being thicker, and more durable so as to hold a higher concentration of active substance (THC). To these film bases only 15 to 25 mg of active ingredients (THC) need to be added according to the desired concentration. In order to make the exemplary recipes possible with the multi-layer and the final thickness, certain problems had to be resolved which will be discussed in greater detail below.

First, the management of the viscosity by way of integration with the mixture of carbon dixoyde (Co2), as carbon dioxide has never been used in dissolving/liquefying aqueous food or pharmaceutical solution. Second, the maximum of active ingredients (THC) that can be integrated into a food film is set by the normal dimension of such a food film on a unit basis, and its thickness. In order to be soluble, ergonomic to consumption, and uniform, a food film is of optimum unit size when the latter has a length of 3 centimeters, a width of 2 centimeters, and a thickness ranging from 50 microns (ideal) to 150 microns (maximum thickness, exceeding this thickness the food film no longer has its intended virtues). Given these optimal dimensions, 150 mg of ingredients may be integrated into a unit food film of this size. Third, the integration of milk proteins (whey), vitamin (ascorbic acid), and botanical extracts (echinacea and guarana). Whey protein powder is very soluble, and contains 90% protein concentration, making it ideal for film integration and much more efficient than WPC (protein concentrate, which is 80% protein). The solution is more creamy and the final product very thick which allows for proteins to be a suitable replacement for some polysaccharides or gums (such as xylose, ribose, glucose (dextrose), mannose, galactose, fructose (levulose), sucrose (sugar), maltose, starches, etc). The use of WPI decreases the amounts of the other “plasticizers” (lycerol, polyethylene glycol (PEG), propylene glycol (PG) and sucrose, which allows to use nearly 12% fewer ingredients. This innovative ingredient's unsuspected properties replace part of the polysaccharides which used to take up an important space in the composition of the final film.

The WPI (whey protein isolate) protein also has a recognized virtue of being an excellent oxygen barrier for given products, which is a critical feature for a food film, which tends to crystallize and crumble when in contact or subjected to changing temperatures and/or oxygen.

In one embodiment, the optimal concentration is 90% WPI/10% glycerol. In some embodiments, the addition of other polysaccharides and standard ingredients, with a decrease of 32% over films of the prior art, the film obtained is stable, durable, flexible, and the humidity (dissolvability) is excellent.

Also, important is the reactivity of the milk protein and ascorbic acid. During testing, this new protein base with botanical extracts (guarana, echinacea) and synthetic caffeine along with vitamin C (ascorbic acid), keeps the same texture and as well as providing a longer lifespan than films of the prior art. In order to prevent ascorbic acid to cause premature crystallization, polysaccharides are replaced with edible gelatin, in some embodiments, “high-bloom cold gelatin”.

In one embodiment, a composition of 50% of the weight of the gelatin film only, and with the integration of the other ingredients provides a film base arranged with WPI and glycerides, which is stable and durable, in addition to enabling the integration of larger dosages of active ingredients (THC).

In some embodiments, a film food with multiple coatings is desired. For instance, some active ingredients (THC) are not able to fit into the same formulation, therefore a triple layer delivery system is utilized which has a 50 micron thick film formulation, allowing for two layers of active agent separated by a layer of food film base.

In order to efficiently superpose the layers, the use of glycerides allows for quick drying between each application of a layer. The use of a neutral separation layer allows for layers containing incompatible ingredients to be used so that a wider variety of ingredients can be used so as to allow more active ingredients to combine in the end and create a synergistic effect not possible in films of the prior art. Without the integration of a base of WPI/Glycerol, this possibility is not conceivable, the film being too thick and exceeding 150 microns. With the improved basic formula and the mixture of carbon dioxide to liquefy the solution and better dissolution (creation of a new material and creation of a new manufacturing process), the film with three coatings is possible and opens up new possibilities that were not accessible using traditional methods.

Now referring to FIG. 2B, a second exemplary active medical ingredients for a multi-layer edible strips is provided, comprising soy lecithin for its emulsifying agent, film lubrication and asset distribution (THC) properties for a better distribution of the active ingredients. In one embodiment, a dosage of 5 mg or less of soy lecithin is desired. Further, the use of modified starches is utilized. For instance, rather than using polysaccharides (or other polymers) or gums, a smaller amount of modified starches in the food film formula optimizes the containment of the active ingredients (THC), keeping them more consistent, and preventing their agglutination, while maintaining greater solubility in water. This new ingredient, never before used in a film matrix, has soluble and homogenizing properties.

In some embodiments, citric acids and tartaric are utilized. The increased solubility of citric, malic, and tartaric acids allows for their addition without weighing down the structure of the multi-layer edible strips (films). That said, to achieve the desired dissolution rate for such a film (1g/6m1) instead of the ideal ratio found at 1g/4m1 (standard film), to allow an absorption as effective and ergonomic for the patient, citric acid is used as a salivary stimulant, which also adds to the flavor. Thus, for a film with a higher dissolution, the absorption time is the same for the patient by stimulating a larger saliva production.

In some embodiments, triacetin is utilized. The film matrix also performs better under the solubility indices with the use of triacetin. (glycerin triacetate3). This ingredient is already used in the medical world as “excipient 4”, but has never, until now, been incorporated into a food film matrix.

In some embodiments, gelatin is utilized. The use of animal and plant-based gelatin, in addition to conventional gums, allows for the dissolution properties of gelatin at 37 degrees Celsius in greater quantity than the gums. The standard gums melt at temperatures that are significantly higher and consequently, the gums cannot be thicker than 35 to 40 micron films. With gelatin, having an index of solubility higher, a melting point lower (37 degrees Celsius), while having the same properties as the often used xanthan or carraghenine gums (they act as plasticizers by consolidating the film), allows the possibility of making films that are 40% thicker without any residues, as is the case of gums thicker than 35 microns. The higher solubility allows for the film to melt in a user's mouth at 37 Celsius.

FIG. 4 is a flow chart illustrating the fabrication process according to an embodiment of the present invention. Referring now to FIG. 4, the fabrication process is described for both formulations/solutions of FIGS. 2A (formulation #1) and 2B respectively (formulation #2). First, solutions are poured into molds of Teflon or non-stick plastic to dry. For the first formulation, the solution is mixed with plasticizers, modified pullulans, and starches previously diluted in hot water. In the second formulation, the active ingredients of a predetermined dosage are prepared and diluted in a solution of deionized water, then heated to 40 degrees Celsius. Next, both solutions are refrigerated down to 5 degrees Celsius then mixed to ensure a liquid consistency and avoid crystallization. In one embodiment, the solutions are mixed at 9500 RPMs. Next, once the liquid has cooled, 50% of the active ingredient solution (B) is poured into solution A (base of the film). Next, at the moment of mixture saturation, the stirring is increased to 12,500 RPMs, and carbon dioxide is injected to liquefy the formula. Also, the pressure is gradually increased from 1 to 4 bars during a period of 5 minutes. Next, once the carbon dioxide is mixed with the solution, the remaining active ingredients are added (solution B) to the mixture. Next, the mixture is stirred at 9000 RPMs until the two solutions of the mixture is completely dissolved into the mixture and the homogenization of the film base. The desired consistency is paste consistency, without crystals. Finally, the mixture is poured into black tinted PET molds with (½″×1″) filling cartridges, and then heated to 350 degrees Celsius on a conveyor.

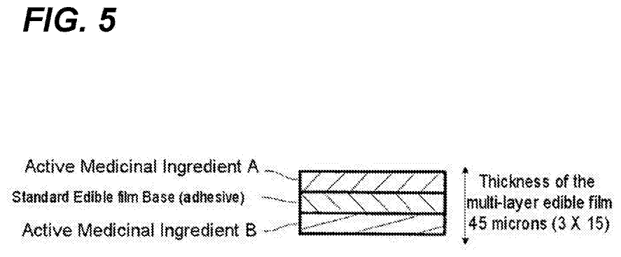

FIG. 5 is a multi-layer edible strip according to an embodiment of the present invention. Referring now to FIG. 5, the multi-layer edible strip is shown. As previously mentioned, the multi-layer edible strip comprises two layers of active medicinal ingredients separated by a standard edible film base adhesive. In some embodiments, the thickness of the multi-layer edible strip is 45 microns, three layers of 15 microns each.

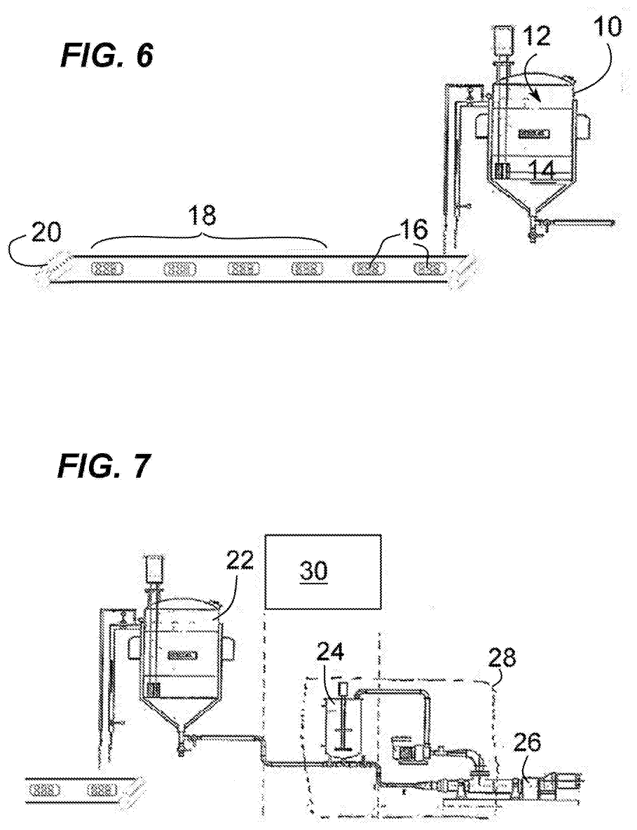

FIG. 6 is a schematic view of the production system of the prior art. In the prior art, basic ingredients of the regular matrix of food film, are placed into a stainless steel tank 10, these are integrated 12 with hydrocollokles and active agents with stirring at 5000 RPM. Homogenization 14 of the mixture occurs, typically for 5 minutes of stirring at 5000 RPM. The solution of poured into non-stick plates 16 with non-stick powder, the plates are dried and heated 18, then processed into molds for cartridge placement 20.

FIG. 7 is a schematic view of the production system according to an embodiment of the present invention. Referring now to FIG. 7, the system comprises integration of the basic ingredients 22 of the film matrix followed by a partial integration of the THC, a deionized water tank 24, a pressure bar measuring valve 26 allowing the Co2 to be gradually blown into the aqueous solution, beyond the theoretical threshold of saturation wherein the Co2 valve 26 is gradually opened to allow insufflation of 4 bars of carbon dioxide pressure. The system further comprises a carbon dioxide feed bottle 28 and a refrigeration unit 30 of the liquid prior to the injection of Co2, to increase the dilution capacity of the mixture and obtain higher concentrations than those obtained by traditional methods.

Although the embodiments described herein representing only two of many possibilities for an attachable and detachable head for a toothbrush. Additionally, the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A multi-layer edible strip comprising:
a first layer of active medicinal ingredients;
a second layer of active medicinal ingredients; and,
a third layer of a food film base, wherein the first layer and the second layer are separated by the third layer.

2. The multi-layer edible strip of claim 1, wherein the active medicinal ingredients include THC.

3. The multi-layer edible strip of claim 1, wherein the multi-layer edible strip is approximately 45 microns to 150 microns.

4. The multi-layer edible strip of claim 1, wherein the third layer of the food film base acts as an adhesive between the first and second layers of active medicinal ingredients.

5. The multi-layer edible strip of claim 2, wherein soy lecithin is used as an emulsifying agent with the THC.

6. The multi-layer edible strip of claim 1, wherein the multi-layer edible strip contains a modified starch.

7. The multi-layer edible strip of claim 1, wherein the multi-layer edible strip contains whey protein isolate and ascorbic acid.

8. The multi-layer edible strip of claim 2, wherein 15 to 25 mg of THC is comprised in the multi-layer edible strip.

9. A multi-layer edible strip consisting of:
a first layer of an ingredients formulation including THC;
a second layer of an ingredients formulation including THC; and,
a third layer of a food film base, wherein the first layer and the second layer are separated by and joined to the third layer.
